SEQUENCE LISTING (12 sequences)

SEQ ID NO: 1 - DNA, 343 bp, Artificial
  Nucleotide sequence encompassing the heavy chain variable region of immunoglobulin produced by hybridoma 2.1
gctcccaggncaactccagcagcctggggctgaaactggtgaagcctggggcttcagtgaagttgtcctgcaaggcttctggctacaccttcaccagctactggatgcactgggtgaagctgaggcctggacaaggctttgagtggattggagagattaatcctagcaatggtggtactaactacaatgagaagttcaagagaaaggccacactgactgtagacaaatcctccancacagcctacatgcaaccagcaacctgacatctgaggactctgcggtcnattactgtacaatcctcaangcttactggggccaanggactcnggtcacagtctctgca

SEQ ID NO: 2 - DNA, 367 bp, Artificial
  Nucleotide sequence encompassing the heavy chain variable region of immunoglobulin produced by hybridoma 6.1
ggggcagagcttgtgaggtcaggggcctcagtcaagttgtcctgcacagcttctggcttcaacattaaagactactatatgcactgggtgaagcagaggcctgaacagggcctggagtggattggatggattgatcctgagaatggtgatactgaatatgccccgaagttccagggcaaggccactatgactgcagacacatcctccaacacagcctacctgcagctcagcagcctgacatctgaggacactgccgtctattactgtaatgcatgggggtactttgactactggggccaaggcaccactctcacagtctcctcagagagtcagtccttcccaaatgtcttccccctcgtctcctgca

SEQ ID NO: 3 - DNA, 334 bp, Artificial
  Nucleotide sequence encompassing the heavy chain variable region of immunoglobulin produced by hybridoma 19.2
ggactgactggtaaggcctgggacttcagtgaagatatcctgcaaggcttctggctacaccttcactaactactggctaggttgggtaaagcagaggcctggacatggacttgagtggattggagatatttaccctggaggtggttatactaactacaatgagaagttcaagggcaaggccacactgactgcagacacatcctccagcactgcctacatgcagctcagtagcctgacatctgaggactctgctgtctatttctgtgcaagatggtggttacgacgggaatggtttgcttactggggccaagggactctggtcactgtctctgca

SEQ ID NO: 4 - DNA, 340 bp, Artificial
  Nucleotide sequence encompassing the heavy chain variable region of immunoglobulin produced by hybridoma 20.1
ggggctgaactggcaagcacctggggcctcagtgaagatgtcctgcaaggcttctggctacacctttactagctacacgatgcactgggtaaaacagaggcctggacagggtctggaatggattggatacattaatcctagcagtggttatactaattacaatcagaagttcaaggacaaggccacattgactgcagacaaatcctccagcacagcctacatgcaactgagcagcctgacatctgaggactctgcagtctattactgtgcaagagggggactattatattactatgctatggactactggggtcaaggaacctcagtcaccgtctcctca

SEQ ID NO: 5 - DNA, 352 bp, Artificial
  Nucleotide sequence encompassing the heavy chain variable region of immunoglobulin produced by hybridoma 45-1
ggagtcaggacctggcctggtggcaccctcacagagcctgtccatcacatgcactgtctctgggttctcattatccagatatagtgtacactgggttcgccagcctccaggaaagggtctggagtggctgggaatgatatggggtggtggaagcacagactataattcagctctcaaatccagactgagcatcagcaaggacaactccaagagccaagttttcttaaaaatgaacagtctgcaaactgatgacacagccatgtactactgtgccagaacctctctctactataggtacggtcctagagctatggactactggggtcaaggaacctcagtcaccgtctcctca

SEQ ID NO: 6 - DNA, 334 bp, Artificial
  Nucleotide sequence encompassing the heavy chain variable region of immunoglobulin produced by hybridoma 47.1
ggaagaagcttggtgcaacctggagggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgttctgatggttactacccgtttgcttactggggccaagggactctggtcactgtctctgca

SEQ ID NO: 7 - DNA, 332 bp, Artificial
  Nucleotide sequence encompassing the heavy chain variable region of immunoglobulin produced by hybridoma 52.1
agcttactgaacctggagggtccctgaaactctcctgtgcagcctctggattcgctttcagtagctatgacatgtcttgggttcgccagactccggagaagaggctggagtgggtcgcaaccattagtagtggtggtagttacacctactatccagacagtgtgaagggccgattcaccatctccagagacaatgccaggaacaccctgtacctgcaaatgagcagtctgaggtctgaggacacggccttgtattactgtgcaagacagacagccaaggtccttgcctggtttgcttactggggccaagggactctggtcactgtctctgca

SEQ ID NO: 8 - DNA, 343 bp, Artificial
  Nucleotide sequence encompassing the heavy chain variable region of immunoglobulin produced by hybridoma 60.1
ggaccgaaatggtcggcctgggacttcagtgaaggtgtcctgcaaggcttctggatacgccttcactaattacttgatanagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagagcctataggtacnactgaggctactggtacttcgatgtctggggcgcagggaccacggtcaccgtctcntca

SEQ ID NO: 9 - DNA, 316 bp, Artificial
  Nucleotide sequence encompassing the heavy chain variable region of immunoglobulin produced by hybridoma 61.6
ggactgagctggtgaacctggggcttcagtgaagatatcctgcaagacttctggatacacattcactgaatacaccatgcactgggtgaagcagagccatggaaagagccttgagtggattggaggtattaatcctaacaatggtggtactagctacaaccagaagttcaagggcaaggccacattgactgtagacaagtcctccagcacagcctacatggagctccgcagcctgacatctgaggattctgcagtctattactgtgcaagactgggacgctactggggccaaggcaccactctcacagtctcctca

SEQ ID NO: 10 - DNA, 328 bp, Artificial
  Nucleotide sequence encompassing the heavy chain variable region of immunoglobulin produced by hybridoma 62.8
ggactgagctggtgaacctggggcttcagtgaagatgtcctgtaaggcttctggatacacattcactgactactacatgaagtgggtgaagcagagtcatggaaagagccttgagtggattggagatattaatcctaacaatggtggtactagctacaaccagaagttcaagggcaaggccacattgactgtagacaaatcctccagcacagcctacatgcagctcaacagcctgacatctgaggactctgcagtctattactgtgcaagagatacggcctggtacttcgatgtctggggcgcagggaccacggtcaccgtctcctca

SEQ ID NO: 11 - DNA, 356 bp, Artificial
  Nucleotide sequence encompassing the heavy chain variable region of immunoglobulin produced by hybridoma 101.5
ggggcctcagtgaacatatcctgcagggctactggctacactttcgataactactggatagagtgggtaaagcagaggcctggacatgcccttgagtggattggagacattttacctggaagtggtactactaactacaatgagaaattcaggggcaaggccacgttcactgcacatacgtcctccaacacaccctaaatgcaactcagcaacctgacatccgaggactctgccttctattactgtgcacggcattactacggtagtacctcatttgcttactggggccaagggactctggtccctgtctctgcagagagtcactccttcccaagtgtcttccccctcgtctcctg

SEQ ID NO: 12 - DNA, 371 bp, Artificial
  Nucleotide sequence encompassing the heavy chain variable region of immunoglobulin produced by hybridoma 107.1
ggagtcaggacctggcctggtggcgccctcacagagcctgtccatcacttgcactgtctctgggttttcattaaccagctatggtgtacactgggttcgccagcctccaggaaagggtctggagtggctgggagtaatatgggctggtggaagcacaaattataattcggctctcatgtccagactgagcatcagcaaagacaactccaagagccaagttttcttaaaaatgaacagtctgcaaactgatgacacagccatgtactactgtgccagaggcggaggctatggtaactacgggtactggggccaagggactctggtcactgtcnctntagagantcagtccttcccaaatgtcttccccctcg

(19) United States
(12) Patent Application Publication
(10) Pub. No.: US 2009/0148460 A1
(43) Pub. Date: Jun. 11, 2009

(54) EXOSOME LIGANDS, THEIR PREPARATION AND USES

(76) Inventors: Alain Delcayre, San Jose, CA (US); Jean-Bernard Le Pecq, Menlo Park, CA (US)

Correspondence Address: NIXON & VANDERHYE, PC, 901 NORTH GLEBE ROAD, 11TH FLOOR, ARLINGTON, VA, 22203, US

(21) Appl. No.: 11/988,440

(22) PCT Filed: Jul. 7, 2006
(86) PCT No.: PCT/IB2006/002907

§ 371 Date: Jan. 8, 2008

Related U.S. Application Data
(60) Provisional application No. 60/697,376, filed on Jul. 8, 2005.

(51) Int. Cl. A61K 39/395; A61K 38/17; A61K 39/00; C07K 16/18; C40B 30/04; A61P 35/00; A61P 31/18; A61P 31/14; A61P 31/04; A61P 37/06

(52) U.S. Cl. 424/152.1; 514/8; 424/178.1; 530/388.2; 506/9

(57) ABSTRACT

The present invention relates to exosome-specific ligands and compositions comprising the same. The invention also relates to methods of generating said ligands and compositions, to methods of using said ligands or compositions, e.g., to block the exosome pathway or to detect and/or characterize exosomes in a sample or subject, as well as to the antigens contacted by said ligands or compositions. The application can be used in experimental, research, therapeutic, prophylactic or diagnostic areas.

The present invention relates to exosome-specific ligands and compositions comprising the same. The invention also relates to methods of generating said ligands and compositions, to methods of using said ligands or compositions, e.g., to block the exosome pathway or to detect and/or characterize exosomes in a sample or subject, as well as to the antigens contacted by said ligands or compositions. The invention can be used in experimental, research, therapeutic, prophylactic or diagnostic areas.

### BACKGROUND

Since their discovery, a growing number of therapeutic applications are in development using exosomes derived from various producing cells, such as dendritic cells (DC), T lymphocytes, tumor cells and cell lines (1,2). For instance, DC-derived exosomes (also designated dexosomes) pulsed with peptides derived from tumor antigens elicit anti-tumor responses in an animal model for the matching tumor (3). Two Phase-I clinical trials using autologous dexosomes for the treatment of lung cancer (4) and melanoma (5), respectively, have recently been completed.

Exosomes derived from tumor cells, cell lines, T cells, are also being assessed as an alternative to dexosomes for the preparation of cancer vaccines (6-9). However, this approach was recently challenged by findings suggesting that these non-DC derived vesicles could also induce immune tolerance (10,11), T cell apoptosis (12,13), metastasis or angiogenesis (14). Given that the protein composition of exosomes depends on the nature of the producing cells, the biological function of exosomes from diverse origin is expected to vary. However, the absence of a standardized and specific method of exosome preparation between laboratories as well as of common exosomes-specific quality control methods to guaranty the classification of vesicles as true exosomes has complicated the interpretation of experimental findings. Indeed, since cells are producing vesicles through various pathways (15), the possibility of contamination of exosomes by other vesicles and the plausible confusion of exosome properties with that, for instance, of vesicles shed from the cell surface (10,16) exist.

Currently, product characterization and quality control for Dexosome therapy relies in part on biomarkers such as CD81 or HLA Class I and II that are common to various biological objects (2). Indeed, these markers are also found on the cell surface as well as microparticles and apoptotic bodies. Clearly, the provision of reagents specific for biomarkers that are unique to exosomes would be a significant improvement for product characterization and QC purposes of this emerging new therapeutic agent. Such reagents would enable to assess and compare the quality and purity of exosomes prepared from various cell types and in different laboratories. They would also be helpful to establish more accurately and definitely the biological functions of true exosomes, as well as to neutralize exosomes in pathological conditions.

Indeed, new studies are now revealing that exosomes may play a critical role in some pathophysiological situations and therefore these vesicles are now also emerging as potential drug targets. In particular, exosomes released by retrovirus-infected and cancer cells display modified properties that could lead to immune evasion of viruses such as HIV and HCV as well as tumors. The most remarkable recent result concerns the hijacking of the exosome pathway by retroviruses. A series of experiments strongly suggest that HIV can not only bud from the plasma membrane in T cells to generate infectious viral particles, but can also bud in macrophages from the limiting membrane of the MVB (17,18, 18b, 18c). This would allow the viruses to enter the exosome pathway. In this model, a viral capside could become inserted inside an exosome and be released by the cell while totally hidden in a vesicle resistant to complement-induced lysis, and be transferred directly and rapidly to other APC. The exosome with its HIV stowaway becomes a diabolical “Trojan Horse”, which, instead of being targeted for elimination by the immune system, takes advantage of its components to spread in disguise and safely multiply. As mentioned in (18b), such exosomes are stable and infectious. Furthermore, this system could be used by other retroviruses and could even allow the spread of retrotransposons (18). Other findings suggest that human hepatitis C virus (HCV) could also use a similar strategy (19). In this case, the exosome might represent the unique mode of spreading for the virus (20). Other viruses like Epstein-Barr could direct viral proteins to exosomes to alter their properties and decrease the immune response (21,22). Similarly, exosomes derived from tumor cells may have altered properties and functions (10). Like virally infected cells, tumor cells can release modified exosomes by incorporating specific proteins (23). By doing so, if these first interesting observations were generalized, tumor exosomes could become a new important component for the understanding of cancer pathophysiology. They could prevent the transfer of antigens to DC causing immunotolerance, transfer to neighbouring cells various proteins such as angiogenic factors to favour their own growth.

Accordingly, it would be particularly useful to develop therapeutic drugs that specifically block the exosome pathway in situation where the pathway leads, for instance, to virus propagation or tumor immune evasion and growth. In addition, exosome-specific ligands or reagents also represent much needed experimental, research and diagnostic tools for the accurate characterization of exosomes and their drug derivatives.

### SUMMARY OF THE INVENTION

The present invention now discloses exosome-specific ligands or reagents, methods for generating such ligands or reagents, as well as their uses, e.g., as therapeutic drugs to block the exosome pathway or as diagnostic or research tools. The invention surprisingly shows that antibodies may be generated against exosomes, which recognize these vesicles and do not bind the exosome-producing cells. Exosome-contacting reagents other than antibodies consisting of compounds reacting with cognate exosome-specific biomarkers and that can potentially block the exosome pathway are also described. Furthermore, the invention describes methods to identify and characterize cognate exosome-specific biomarkers. Finally, methods to generate biomarker derivatives and to use biomarkers and said derivatives as therapeutic drug to block the exosome pathway are also described.

Exosome-contacting reagents that react specifically with exosomes but not with exosome-producing cells are very much needed and useful for at least two main purposes. First, the systematic and accurate characterization of exosomes for research purpose and clinical use is required. Indeed, reagents used so far to characterize exosomes are antibodies that recognize known markers found on cells as well as other cell-derived vesicles. Exosome-specific reagents will provide a more rigorous means for the quality control of exosome-based products tested in the clinics. Second, highly specific exosome reagents represent therapeutic agents to treat, for instance, pathophysiological conditions requiring blocking of the exosome pathway.

The invention relates to a method of generating an exosome-specific antibody, comprising:

1) preparing vesicles, preferably exosomes from a non-human animal with a given genetic background;
2) immunizing a non-human animal with said vesicles, wherein animals for immunization and preparation of vesicles share the same or very similar genetic background; and
3) collecting, from said immunized animal, antibodies reacting with the vesicles of step 1 or corresponding producing cells.

The antibodies can be isolated directly from animal serum for the preparation of polyclonal antibody. Monoclonal antibodies may also be prepared from these animals using traditional approaches including generation of hybridomas or isolation of single antibody-producing cells by methods such as SLAM. In a specific embodiment, step 3) comprises the steps of a) collecting lymphocytes from the immunized animal and b) identifying and isolating single antibody-producing cells producing antibodies reacting with the vesicles of step 1.

The specificity of the antibodies may then be assessed in various assays, particularly binding assays against different material (e.g., the exosome-producing cell).

Preferably, the vesicles of step 1) are obtained from mammalian exosome-producing cells. These cells may be normal, transformed, tumoral or infected with a virus or microbe. More preferably, said mammalian exosome-producing cells are murine cells.

In another embodiment, the invention relates to a method of isolating exosomes specific ligands- or reagents, comprising:

1) preparing vesicles, preferably exosomes,
2) screening libraries of compounds reacting with said vesicles, and
3) selecting exosome binding ligands from step 2 that do not react with the exosome-producing cells or with other biological objects, preferably cells.

Compound libraries include peptide/polypeptide-, glycolipid- or nucleotide-based libraries as well as small molecules libraries. Preferably, the library is a recombinant or natural antibody library. Non-antibody exosome-contacting compounds may be modified according to known methods of the art to generate improved exosome blocking agents. These include notably mutation of binding site or fusion to partner permitting polymerization, preferably dimerisation, of exosome-contacting reagent. Such modifications are expected to increase the affinity of the reagents for exosomes, thereby improving blocking efficiency. Alternatively, exosome-contacting reagents may be chemically modified or fused to partner permitting faster exosome elimination.

The invention also relates to a method of screening active compounds, the method comprising contacting in vitro exosomes from infected cells in the presence of T cells and a candidate compound, and assessing the ability of the candidate compound to neutralize infection of T cells. More preferably, the exosomes are obtained from a virus-infected cell, e.g., an HIV infected cell. Even more preferably, the exosomes are obtained from infected macrophages.

The invention also relates to the use of exosome-specific ligands to identify exosome-specific biomarkers.

The invention also relates to a method of identifying exosome-specific biomarkers comprising:

1) preparing vesicles, preferably exosomes from a non-human animal with a given genetic background;
2) immunizing a non-human animal with said vesicles, wherein animals for immunization and preparation of vesicles share the same or very similar genetic background;
3) obtaining antibodies reacting with the vesicles of step 1, and
4) identifying and isolating the cognate biomarkers using antibodies of step 3.

Biomarkers identified by this method may be any protein, for example a receptor or an enzyme, or compounds other than polypeptides, such as glycolipids, polysaccharides and nucleotides. Said biomarker may also be a tumor, a viral or a microbial antigen, depending on the nature of the cells used to produce exosomes for immunization.

The invention further concerns the biomarkers targeted by exosome specific reagents, and their use for research, diagnostic and therapeutic applications. The invention also concerns a composition comprising said biomarkers and a pharmaceutically acceptable excipient or carrier.

In another embodiment, the invention relates to the isolation and design of new exosome specific reagents using exosome-specific biomarkers. Indeed, biomarkers instead of exosomes may now be used to isolate new exosome-contacting reagents using the methods described above. Alternatively, because of their exosome-specificity, biomarkers or their modified forms may themselves be used as exosome-contacting reagents. Biomarker modification may include mutagenesis to abolish undesirable biological activities or increase exosome-contacting affinity, fusion to polypeptide permitting polymerization, preferably dimerisation, of the biomarker, fusion to entities improving exosome elimination.

The invention also relates to a method of identifying an exosome-specific biomarker, comprising:

1) preparing vesicles, preferably exosomes from a subject;
2) injecting the vesicles to the said subject;
3) optionally, collecting lymphocytes from the subject; and
4) identifying and isolating single antibody-producing cells producing antibodies reacting with the vesicles of step 1.
5) preparing the antibodies produced by antibody-producing cells of step 4
6) identifying and isolating the cognate biomarkers using antibodies of step 5.

Preferably, said subject is a patient and exosome-producing cells are tumor cells or virus or microbe infected cells.

The invention also relates to a method of producing an antibody response in a subject against an exosome, comprising:

administering to a subject in need thereof an exosome-specific biomarker as disclosed above, or as obtained by a method as disclosed above.

The invention further concerns isolated exosome-contacting reagent-producing cells, and their use for reagent production, and the reagent produced by said cells. The invention also concerns a composition comprising either said isolated exosome-contacting reagent-producing cells or said reagent produced by said cells and a pharmaceutically acceptable excipient or carrier.

A further object of this invention relates to methods for neutralizing exosomes in a subject, the method comprising administering to a subject in need thereof a neutralizing-effective amount of an exosome-specific ligand, said administration causing a neutralization of exosomes. The subject may be any mammalian subject, particularly a human subject. The subject may suffer from any pathological condition that could benefit from a blocking of the exosome pathway, including an infectious disease, a cancer, an autoimmune disease, an inflammatory disease, transplantation, etc.

Another object of this invention relates to compositions comprising an exosome-specific ligand and a pharmaceutically acceptable carrier or excipient.

The invention also relates to antibodies, or a fragment or derivative thereof having substantially the same antigen specificity, wherein said antibodies selectively bind an exosome. Specific examples of such antibodies or fragments include any antibody, or fragment or derivative thereof, which comprises a heavy chain variable polypeptide sequence encoded by a nucleotide sequence selected from SEQ ID NOs: 1 to 12. The invention also encompasses any chimeric polypeptide comprising an antibody, or fragment or derivative thereof, as defined above.

A further aspect of this invention resides in exosome-specific ligands, wherein said ligands comprise an exosome-specific ligand-binding domain fused to a dimerization or multimerization domain or compound, in particular to an Fc portion of an immunoglobulin.

A further aspect of this invention resides in exosome-specific ligands, wherein said ligands comprise a protein, or a domain thereof, that selectively binds an exosome-specific marker, fused to an exosome-specific antibody or a fragment or derivative thereof.

The invention also relates to the use of exosome-specific ligands to detect exosomes in vitro, ex vivo or in vivo. Detection may be performed in vitro or ex vivo in any isolated sample, such as a biological fluid, a tissue, a biopsy, etc. The detection may also be performed directly in vivo, e.g., using a labeled exosome-specific ligand.

The invention further includes methods of characterizing an exosome preparation, comprising contacting an exosome preparation with an exosome-specific ligand and determining the presence and/or amount and/or nature of exosomes in said preparation.

The invention also relates to the use of an exosome to stimulate B cells in vitro, ex vivo or in vivo, or to manufacture a medicament for stimulating a B cell response in a patient.

The invention also relates to a method of stimulating B cells in vivo, comprising administering to an animal or a subject in need thereof an amount of exosomes sufficient to cause a stimulation of B cells in said animal or subject.

The invention also relates to a method of stimulating B cells in vitro, comprising culturing lymphocytes in vitro in the presence of exosomes, under conditions allowing B cell stimulation. More preferably, the lymphocytes are obtained from an animal or subject.

Preferably, the subject is a patient and the exosomes are obtained from tumor cells or virus or microbe infected cells.

### LEGEND TO FIGURES

FIG. 1: ELISA detecting anti-Exosome antibody in sera of exosome-immunized mice.

FIG. 2: FACS analysis of exosomes and exosome producing cells with anti-exosome antibodies.

FIG. 3: Stimulation of B cells by exosomes

### DETAILED DESCRIPTION OF THE INVENTION

### Definitions

Within the context of this invention, the term “exosome” refers to externally released vesicles originating from the endosomic compartment or cells, including tumor cells and immune cells, particularly antigen presenting cells, such as dendritic cells, macrophages, mast cells, T lymphocytes or B lymphocytes. More specifically, such vesicles are of endosomal origin and are secreted in the extracellular milieu following fusion of late endosomal multivesicular bodies with the plasma membrane (1). Methods of producing, purifying or using exosomes for therapeutic purposes or as research tools have been described in WO99/03499, WO00/44389 and WO97/05900, incorporated therein by reference.

An “exosome-contacting reagent” or “exosome-specific ligand or reagent” designates any compound that binds an exosome and essentially does not bind the cell from which said exosome derives. Although non-specific binding to other objects may not be excluded, such non-specific binding can be discriminated from the specific binding to exosomes. Exosome-specific ligands or reagents (ESL) thus typically bind compounds or structure (e.g., biomarkers) that are specifically or predominantly present at the surface of exosomes, and essentially absent from other object or cells. ESL may be derived from diverse classes of natural and synthetic compounds, including notably antibodies, dimerized cognate ligands or receptors, small molecules, etc.

A typical example of ESL includes antibodies, as well as fragments and derivatives thereof having substantially the same antigen specificity. Such fragments include Fab, F(ab′)2, CDR, variable regions, etc. Such derivatives include single chain antibodies, humanized antibodies, human antibodies, bi-functional antibodies, etc.

In a specific embodiment of this invention, the ESL is a monoclonal antibody or a derivative or fragment thereof, even more preferably an antibody, or fragment or derivative thereof, comprising a heavy chain variable polypeptide sequence encoded by a nucleotide sequence selected from SEQ ID NOs: 1 to 12.

In a further embodiment of this invention, the ESL is a protein, or a domain thereof, that selectively binds an exosome-specific marker. A preferred example of such a protein is lactadherin, or a fragment thereof comprising at least the C1 and/or C2 domain of lactadherin, or a functional equivalent thereof. Functional equivalents include other proteins or protein domains containing C1 and/or C2-like domains, such as neuropilin or del-1, for instance, as well as variants of these proteins, e.g., mutants thereof having increased affinity or specificity for exosomes. Such mutants typically contain 1 or 2 or 3 or 4 or 5 mutated amino acid residues, i.e., replaced, deleted or inserted within the sequence. The sequence of lactadherin, neuropilin and del-1 is available from the literature and database.

The ESL of this invention may be in the form of monomers or multimers, to further increase their affinity and therefore specificity towards exosomes. In this regard, in a particular object, the ESL is a chimeric polypeptide comprising an ESL-binding domain fused to a dimerization or multimerization domain or compound, in particular to an Fc portion of an immunoglobulin.

In a further embodiment of this invention, the ESL comprises several ESL-binding domains. In this respect, the ESL preferably comprises a protein, or a domain thereof, that selectively binds an exosome-specific marker, fused to an exosome-specific antibody or a fragment or derivative thereof.

The ESL may be labeled, e.g., using any conventional label such as fluorescence, luminescence, enzymatic, biochemical or a radioactive labels.

The ESL of this invention shall specifically bind exosomes, as discussed above, thereby allowing exosome detection and characterization in any sample or in vivo. Furthermore, in a preferred embodiment, such ESL may be used to neutralize exosomes.

In this respect, the term “neutralizing” designates, within the context of this invention, reducing, blocking, inhibiting or preventing the activity of exosomes, e.g., their ability to freely circulate in fluids, to transport and/or present molecules and/or to efficiently interact with and/or penetrate into cells. Such neutralization may be partial and temporary. In a preferred embodiment, the ESL neutralizes exosomes by aggregating these vesicles, leading to their clearance. Such an aggregation can be obtained using multimeric or multifunctional ESL, as disclosed above.

The present invention discloses antibodies that can be used to block the exosome pathway and identify exosome-specific biomarkers. The invention further discloses methods to isolate ESL other than antibodies and methods to generate biomarker derivatives that can also be used to block the exosome pathway. The invention also discloses methods to stimulate B lymphocytes in an animal or an individual as well as in ex vivo or in vitro cultures. This invention can be used in experimental, research, therapeutic, prophylactic or diagnostic areas.

The present invention stems from the discovery of unexpected B lymphocyte activation and antibody responses in situations where the object used for immunization, for instances an exosome, is derived from cells of identical or similar genetic background than the non-human animal used for immunization. More particularly, the invention shows that these antibodies react specifically with exosomes and thereby identify novel biomarkers of this subcellular compartment. Generally, antibody responses in animals emanate from exposure to a foreign or non-self antigen. Antibodies to exosomes can therefore be generated by immunizing non-human animals in a xenogenic or allogenic manner. In the first case, the cells producing exosomes are derived from a different species than that of the immunized animals. Theoretically, all antigens are foreign and antibodies against all exosome antigenic entities may be obtained. In the second case, the cells producing exosomes are derived from the same species than that of the immunized animals; however, the two differ in that they express different subtypes of polymorphic polypeptides. Here, an antibody response restricted against these polymorphic antigenic entities is expected since all other antigens are self antigens. As expected, both xenogenic and allogenic immunization with exosomes yield very strong antibody responses. By contrast, syngeneic immunization where cells producing exosomes and immunized animals share identical genetic background, i.e. cells are derived from the same strain than the immunized animal, and autologous immunization, where the cells producing exosomes are derived from the same animal used for immunization, are not expected to produce any anti-exosome antibody responses. In this situation, all antigens are recognized as self-antigens unless the exosome content has been modified to expose antigenic determinants not present during B cell negative selection and development process or unless tolerance has been broken.

For obvious reasons standard methods for raising antibodies use xenogenic or allogenic immunization procedures. These responses are mainly directed against dominant antigenic entities that are also present in other cell compartments, notably at the surface of cells. To date, only one antigen, i.e. Lactadherin, appears to be almost exclusively expressed on exosomes. A domain called C1C2 is responsible for this specific subcellular distribution and methods for the preparation of vesicles bearing chimeric proteins that contain the C1C2 exosome targeting domains and their use are described in PCT/EP (WO 03/016522). It turns out that earlier studies suggested that Lactadherin may constitute a potential drug target in breast cancer (24). More recently, a role of Lactadherin in stimulating VEGF-mediated vascularization was reported (25). Hence, Lactadherin may in fact represent the first example of drug target for anti-exosome antibody therapeutic. However, because Lactadherin expression is restricted to specific tissues, i.e. mammary gland (24) and some tumors (our unpublished data), it does not constitute a true or universal exosome biomarker. In fact, prior to the instant invention, no antibody reacting with a non-polymorphic antigenic entity unique to exosomes had ever been described. The present invention reveals that antibody responses to exosomes can be induced using a syngeneic immunization procedure and that this procedure favours raising antibodies directed to specific new markers on exosomes.

In addition to the identification of new biomarkers, the invention presents the advantage of providing new means to accurately characterize exosome-based pharmaceutical preparations and potentially, block specifically the exosome pathway.

Furthermore, the invention also provides a novel method to stimulate B lymphocytes in vitro and in vivo.

### Method for Inducing Exosomes-Specific Antibody Responses

In this method, antibodies are raised when vesicles, preferably exosomes, derived from cells of a given genetic background are introduced into an animal with the same genetic background than the cells used to prepare exosomes. The production of antibody may be evaluated by testing the serum of immunized-animal by standard approaches. The preparation of antibody may be accomplished by affinity purification of antibodies from the serum for polyclonal antibodies or by isolating antigen-specific antibody producing cells for monoclonal antibodies. Alternatively, monoclonal antibodies may be prepared using the various known approaches of monoclonal antibody preparation from immunized animal such as Hybridoma screening (26) and SLAM (27).

Immunogens, preferably exosomes, are prepared from cell cultures and purified by centrifugation on a sucrose gradient (U.S. Ser. No. 09/780,748). Exosome-producing cells include any cell, preferably of mammalian origin, that produces and secretes membrane vesicles of endosomal origin by fusion of late endosomal multivesicular bodies with the plasma membrane (1,2). Cells from various tissue types have been shown to secrete exosomes, such as dendritic cells, B lymphocytes, tumor cells, T lymphocytes and mast cells, for instance. Methods of producing, purifying or using exosomes for therapeutic purposes or as research tools have been described for instance in WO99/03499, WO00/44389, WO97/05900, incorporated therein by reference. Preferred exosome-producing cells of this invention are mammalian tumor cells, virus-infected cells, B and T lymphocytes and dendritic cells, typically of murine or human origin. In this regard, the cells are preferably immortalized dendritic cells (WO94/28113), immature dendritic cells or tumor cells (WO99/03499).

The cells may be cultured and maintained in any appropriate medium, such as RPMI, DMEM, AIM V etc., preferably protein-free media to avoid contamination of exosomes by media-derived proteins. The cultures may be performed in any suitable device, such as plates, dishes, tubes, flasks, etc. Alternatively, exosomes may be prepared from serum of individuals.

Antibodies are raised following inoculation of exosomes to animals, preferably in a syngeneic manner, i.e. where immunized animals have the same genetic background than the cells used to prepare exosomes. Alternatively, exosomes derived from cells belonging to the same species but with a different subtype of polymorphic polypeptides may be used when the expression of the major polymorphic polypeptides such as MHC I and II molecules is downregulated. In yet another method, anti-exosomes specific antibodies may be raised following inoculation of exosomes to animals in xenogenic or allogenic manner after animals have been tolerized to foreign or polymorphic polypeptides. Tolerization may be achieved by method known of the art including repeated exposure of the animals to the said foreign and polymorphic polypeptides.

The antibodies may be polyclonal or monoclonal. Animals can be from various species, including mice, rodents, primates, horses, pigs, rabbits, poultry, etc. Preferred animals are mice.

The inoculum composition generally further comprises a pharmaceutically acceptable excipient or vehicle, such as a diluent, buffer, isotonic solution, etc. The composition can further comprise an adjuvant.

Administration of inoculum can be performed by various routes, such as by systemic injection, e.g., intravenous, intramuscular, intra-peritoneal, intra-tumoral, sub-cutaneous, intra-splenic, intra-nodal etc.

The isolation of exosome-specific monoclonal antibodies is performed by standard methods of antibody repertoire screening, including ELISA of supernatant from antibody-producing cell cultures or sorting of cells expressing cell-surface antibodies. The same exosomes used for immunization is also used as vehicle for such screening. Antibody-producing cells are preferably primary or immortalized B lymphocytes. Immortalization may result from transformation of B lymphocytes with transforming agents including viruses and oncogenes or from fusion with immortalized cells to prepare hybridoma. Antibody-producing cells may also be libraries of prokaryotic or eukaryotic recombinant cells expressing antibodies, preferably human antibodies. B lymphocytes may be derived from immunized animals, preferably transgenic animals expressing human immunoglobulins.

The invention also relates to the isolation of soluble antibodies that may be derived from blood of immunized animals or a fraction thereof. Soluble antibodies may also be derived from any expression system producing recombinant antibodies individually or as pools or libraries of recombinant antibodies with various antigen specificities. Alternatively, other libraries of compounds that can potentially react with biological objects or molecules, i.e. peptide/polypeptides, glycolipids, nucleotides or small molecules may also be used for the screening of ESL. For this screening, exosome contacting reagents are first isolated by standard binding procedure, such as affinity-chromatography. ESL are then selected for not binding to exosome-producing cells in a second step.

### Anti-Exosome Antibodies

The invention now discloses twelve novel antibodies and antibody-producing cells that were obtained by the immunization method described above. These antibodies can potentially be used for research, diagnostic or therapeutic uses.

The nucleotide sequences of the variable region of antibody heavy chains are depicted (SEQ ID 1 to 12). These antibodies were produced by hybridoma after fusion of the cell line SP2/0 with spleen cells of mice immunized with syngenic exosomes. Hybridoma producing these antibodies were identified and isolated by screening hybridoma supernatants in standard ELISA using exosomes as antigen and sorting using a fluorescent activated cell sorter (FACS). The antibodies of the present invention were selected for the unique properties of reacting with a marker on exosome that is absent on the surface of the exosome-producing cells. Therefore, these antibodies identified more likely novel exosome-specific biomarkers.

The antibodies of the present invention may be produced directly from hybridoma. Alternatively, the nucleotide sequences encoding at least the variable regions of heavy and light chains of immunoglobulins produced by the hybridomas may be cloned into expression vectors for their production as full-length or fragments of recombinant antibodies.

The invention also relates to a polypeptide composition comprising at least 50% primary structure identity with the polypeptides encoded by SEQ ID 1 to 12. Identity may be determined according to various known techniques, such as by computer programs, preferably the CLUSTAL method. More preferably, the polypeptide composition has at least 60% identity, advantageously at least 70% identity with the polypeptides encoded by SEQ ID 1 to 12. Such polypeptide variant (or functional equivalent) should retain the ability to react specifically with exosomes and not with the surface of the producing cells. This property may be verified as described in the example 2, by comparative FACS staining of both exosomes and the exosome-producing cells. Possible variations include amino acid deletion(s), substitution(s), mutation(s) and/or addition(s). Sequences of antibody and related polypeptide composition may be further manipulated by recombinant DNA techniques to increase the affinity of the antibody for its target antigen, a process known as antibody maturation, or to humanize the antibody for therapeutic applications. Such variants may also be produced to design polypeptides with improved pharmacokinetics properties.

The invention also concerns a composition comprising either said isolated antibody-producing cells or said antibodies produced by said antibody-producing cells and a pharmaceutically acceptable excipient or carrier.

### Exosome-Specific Antigens or Biomarkers

In an other embodiment, the invention concerns the antigens or biomarkers targeted by exosome-specific antibodies and their use for research, diagnostic and therapeutic applications. These antigens may be non-characterized compounds and antibodies against said antigens are necessary in order to identify the function of said antigens and to characterize them. Methods utilizing antibodies to characterize antigens include notably standard immunoprecipitation and immunoblotting procedures that enable antigen isolation. Isolated antigens may then be identified by various methods known of the art for revealing their composition. The antigen recognized by the antibodies described above can be any protein, for example receptors or enzymes, or compound other than polypeptides, such as glycolipids, polysaccharides and nucleotides present on exosomes. These antigens may also be novel tumor antigens, viral antigens, and microbial antigens.

Obviously, once discovered, exosome-specific biomarkers may be used in their purified form or in association with exosomes to identify new ESL using known methods of compound screening. Such compound may be derived from polypeptides, glycolipids, polysaccharides, nucleotides or small molecules libraries. Alternatively, the natural cognate ligands of exosome-specific biomarkers also constitute new potential ESL. Finally, the biomarkers themselves or part thereof may become ESL. Indeed, because of their restricted expression profile to exosomes, it is expected that biomarkers may contain exosome targeting domains. The identification of such domain and their usage to target desired entities to exosomes has been described in PCT/EP (WO 03/016522). More specifically, the C1C2 domain of Lactadherin mediates the specific targeting to exosomes of any polypeptide fused to it. Methods to target antigens, cytokines and other polypeptide to exosomes and their applications have also been described in PCT/EP (WO 03/016522). This method can now be used to block the exosome pathway. For instance, a chimeric construct comprising the C1C2 domain of Lactadherin or its variants fused to the Fc fragment of an immunoglobulin may have similar consequences than an anti-exosome antibody for the blocking or elimination of circulating exosomes.

Hence the invention concerns the biomarkers or fragments thereof and their use to either identify new ESL or as part of ESL. The invention also concerns a composition comprising said biomarkers and a pharmaceutically acceptable excipient or carrier.

Exosome-Pathway Neutralization

As exosome function unveils, a growing body of information support that it may be advantageous to block or neutralize the exosome pathway in specific pathophysiological situations. The latter include notably cancer and microbial infection with for instances HIV and HCV. Because of its role in the immune response, blocking the exosome pathway may also be desirable during inflammation to restore immune unbalance leading to autoimmunity, tolerance or immuno-suppression. In this respect, the invention concerns the use of ESL as therapeutic agents.

The invention more particularly relates to the use of an ESL, as defined and disclosed therein, in the preparation of a pharmaceutical composition for the prevention or treatment of a pathophysiological condition requiring blocking of the exosome pathway, including an infectious disease, a cancer, an autoimmune disease, an inflammatory disease, transplantation, etc.

In this regard, a particular object of this invention resides in a method for neutralizing a pathological immune response in a subject, the method comprising administering to a subject in need thereof a neutralizing-effective amount of an ESL, said administration causing a neutralization of said pathological immune response. The pathological immune response may be selected from an allergic response, an inflammatory response, including host graft response following transplantation, and an autoimmune response.

A particular object of this invention also relates to a method for neutralizing or treating (viral or microbial) infection in a subject, the method comprising administering to a subject in need thereof an amount of an ESL effective to neutralize exosomes in said subject, said administration causing a neutralization or treatment of said infection.

A further particular object of this invention is a method for neutralizing viral dissemination in a subject, the method comprising administering to a subject in need thereof an amount of an ESL effective to neutralize exosomes in said subject, said administration causing a neutralization of said viral dissemination.

As discussed above, the virus may be selected from an immunodeficiency virus and a hepatitis virus.

A more specific embodiment of this invention is a method for neutralizing HIV infection or dissemination in a subject, the method comprising administering to a subject in need thereof an amount of an ESL effective to neutralize exosomes in said subject, said administration causing a neutralization of HIV infection or dissemination. In a particular embodiment, the method is used in combination with other anti-viral therapies, e.g., tri-therapy.

A further particular object of this invention is a method for treating cancer (e.g., by reducing cancer immune evasion) in a subject, the method comprising administering to a subject in need thereof an amount of an ESL effective to neutralize exosomes in said subject, said administration causing a treatment of said cancer.

The ESL or reagent may be an exosome-specific antibody as described above. Alternatively, they may be biomarker derivatives, natural cognate ligands of biomarkers or derived from libraries of compounds other than antibodies also as described above.

Exosome pathway blocking may be improved by modifying exosome contacting reagent to either improve their affinity for their target or permit efficient elimination of exosomes. It could also lower toxicity or side effects when biomarkers derivatives are used as blocker. For instance, as mentioned above, Lactadherin is a biomarker of exosomes that can potentially be used as an ESL to block the exosome pathway. Lactadherin contains a RGD site mediating binding to its cognate integrin receptors (24). A point mutation modifying the RGD site into RGE abolishes this binding and therefore the biological activity of Lactadherin. Such mutant would maintain its ability to contact and therefore to potentially block exosomes. Another modification of Lactadherin could aim at increasing its affinity for exosomes. This may be achieved for instances by fusing the C1C2 domain of Lactadherin to the Fc domain of immunoglobulin. This fusion enables dimerization of Lactadherin resulting in high binding efficiency. Finally, another example of modification that may improve the blocking of the exosome pathway is fusion of two exosome-contacting reagents. For instances, the heavy chain of the F(ab′)2 fragments of antibodies reacting specifically with exosomes may be fused to the C1C2 domain of Lactadherin. When such fusion is performed downstream the dimerization site of immunoglobulin heavy chains, it yields tetrameric compounds containing two antibody binding sites and two C1C2 domain of Lactadherin. It is anticipated that the binding affinity and specificity of this tetrameric reagents will be greatly increased, thereby improving the efficacy of exosome pathway neutralization.

Interference with the exosome pathway or exosome neutralization using exosome contacting reagents and derivatives as described above may be provided by injection of the reagents to a subject.

In another embodiment the biomarkers may be used in a purified form or associated with exosomes to induce an antibody response in a subject. Indeed, biomarker or autologous exosomes comprising the said biomarker may be injected to a subject to generate neutralizing anti-exosomes antibodies directly in the said subject. In this respect, the use of biomarkers or exosome-containing biomarkers may be therapeutic and also prophylactic. Exosome-containing biomarkers may be derived from immortalized or tumor cells. Exosomes may also be derived from cells infected with viruses or microbes. Exosomes may be derived from in vitro culture of subject cells or subject body fluid including serum. Immortalized, tumor or infected cells may be isolated directly from the subject or prepared in vitro from normal cells.

### B Lymphocyte Stimulation

The invention now discloses methods to stimulate B lymphocytes that stem from the unexpected B cell responses obtained by the immunization method described above. These methods can potentially be used for research, diagnostic or therapeutic uses.

In these methods, B lymphocyte stimulation is induced when vesicles, preferably exosomes, derived from cells of a given genetic background are introduced into an animal with the same genetic background than the cells used to prepare exosomes. Cell stimulation may be evaluated by assessing the number and frequency of B lymphocytes in lymphoid organs or in the periphery of immunized-animal by standard approaches. B lymphocytes may also be stimulated in vitro when the said vesicles are added to ex vivo cultures containing B lymphocytes. Cell cultures may comprise mixed cell populations or purified B lymphocytes cultured as pooled or single cells. Cell stimulation may be evaluated by measuring H³-thymidine incorporation by standard approaches.

A particular object of this invention is a method of adjuvant therapy in a subject, the method comprising administering to a subject in need thereof an amount of an exosome in said subject, said administration causing the proliferation of B lymphocytes. Such adjuvant therapy may be performed using exosomes alone or in combination with an antigen.

A further particular object of this invention is a method to expand B lymphocyte cultures in vitro comprising incubating cell cultures with exosomes, said incubation causing the proliferation of B lymphocytes. Antigen-specific B cell proliferation may be achieved by supplementing the cultures with an antigen. Such stimulation may for instance be used to expand pooled or single cell cultures containing antibody-producing cells from immunized-animal or a subject. B lymphocyte proliferation may facilitate isolating antigen-specific antibody-producing cells required for the preparation of monoclonal antibodies using the various known approaches of monoclonal antibody preparation from immunized animal such as Hybridoma screening (26) and SLAM (27).

Further aspects and advantages of the present invention will be disclosed in the following examples, which shall be considered as illustrative and not limiting the scope of this application.

### EXAMPLES

### Example 1

### Induction of Anti-Exosome Antibody Responses Upon Syngeneic Immunization

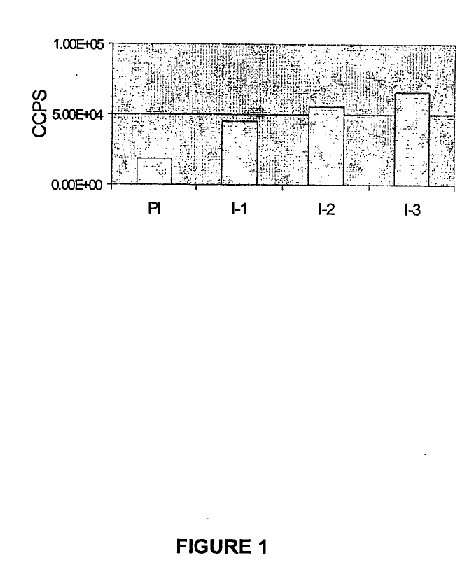

The murine lymphoblastoid cell line YAC was selected for production of exosomes as it is negative for the major polymorphic markers MHC I and II. Theoretically, most polypeptides on YAC exosomes are expected to be self antigens regardless of the strain of mice used for immunization. YAC cells were expanded into 1-liter spinner flask in ADCF media (Hyclone), a protein-free media for large-scale production of exosomes. Five-day cell culture supernatant was transferred into 250-ml centrifuge bottles and spun 5 min at 2000 rpm to pellet cells. The supernatant was then filtered through 0.2 μm filter and concentrated to 100 ml using a fiber cartridge with a 500 KD size cut-off. Concentrated supernatant was then layered onto a 30% sucrose cushion and spun under 100,000 g for 1 hour 15 min at 4° C. Gradient interface was collected and sucrose was removed by PBS-diafiltration using a 500 KD fiber cartridge as above. Multiple subcutaneous injections of 5 μg of exosomes in PBS were performed in mice at 15-day intervals. Blood sample were collected between injections to assess antibody responses. Anti-exosomes antibodies were detected by standard ELISA using YAC exosomes as antigen. For the ELISA, 500 ng YAC exosomes in PBS was coated to the wells of a microtitration plate overnight at 37° C. Blocking buffer containing 0.05% Tween-20 in PBS was added to the wells for one hour at room temperature (RT) to saturate the remaining free binding sites. Wells were then incubated for one hour at RT with serum of immunized mice at a dilution 1/500 in Blocking buffer. After washing the wells three times with Blocking buffer, bound antibodies were detected using a 1/10000 dilution of horse-radish peroxidase (Jackson ImmunoResearch) and an ECL substrate (Amersham). The results of this ELISA are shown in FIG. 1.

Results: A signal above background was detected after multiple immunizations with exosomes (1-1 to 1-3). Background was determine as the signal detected when using serum collected pre-immunization (P-I).

Conclusion: Although the exosomes used for immunization are expected to contain only self antigens, an antibody response was detected. Despite the multiple injections and the large quantity of material injected, the titer of anti-exosome antibodies in the serum of immunized mice remains very low. This response profile is in line with the non-classical immunization procedure used in the present invention.

### Example 2

### Specificity of Anti-Exosome Antibodies

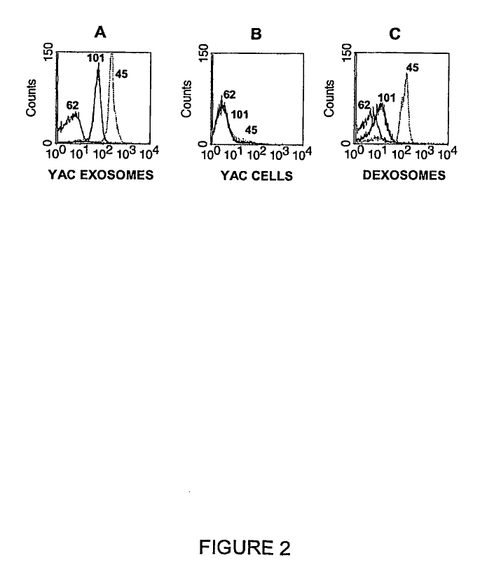

Spleen cells of mice immunized as described in Example 1 were fused to SP2/0 to produce hybridoma. Hybridomas were then screened and selected for production and secretion of anti-exosome antibodies by ELISA also as described in Example 1. Antibodies of the IgM subtype produced by the Hybridoma clone 45, 101 and 62 (negative control) were purified from culture supernatant by diafiltration using a fiber cartridge with a 500 KD size cut-off. The purity of diafiltered and concentrated fractions was verified to be at least 90% by SDS-PAGE analysis. The purified antibodies were used to stain exosomes coated on 1-μm beads as well as exosome-producing cells. Staining was detected by FACS analysis using a secondary anti-mouse IgM conjugated to a fluorophore. The results of this analysis are shown in FIG. 2.

Results: A shift of fluorescence was detected when YAC exosome-coated beads were incubated with IgM 45 and 101, whereas no fluorescence was detected with the negative control antibody IgM 62 (FIG. 2, panel A). In contrast, IgM 45 and 101 did not bind to YAC cells as cell staining did not yield a fluorescent shift with any antibody tested (FIG. 2, panel B). Furthermore, IgM 45 and 101 were also found to react with exosomes from a different cell type and species since a shift of fluorescence was detected with these antibodies when beads were coated with human dendritic cell-derived exosomes or dexosomes (FIG. 2, panel 3).

Conclusion: This data suggest that IgM 45 and 101 recognize antigenic determinant(s) expressed on the exosomes used for immunization but not on the surface of the cells used to produce the said exosomes. Moreover, this(these) antigenic determinant(s) is(are) conserved in exosomes derived from different cell types and species. Hence they most likely represent novel entities or biomarkers specific to the exosome compartment.

### Example 3

### In Vivo and In Vitro Stimulation of B Cells by Exosomes

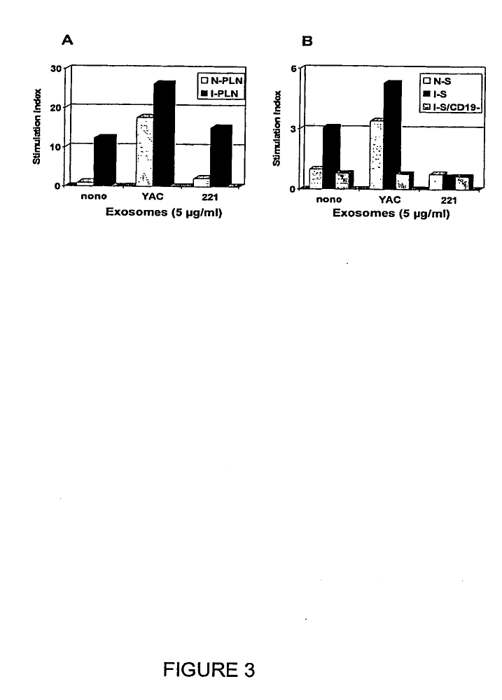

Spleen and popliteal lymph nodes of mice immunized as described in example 1 were collected and cells were counted using an hematocytometer and stained with PE-conjugated anti-CD19 antibody to determine the percentage of B cells. Cells were also plated into the wells of 96-well tissue culture plated at 2×10E5 cells/wells in RPMI/15% FCS supplemented or not with exosomes. Spleen cell samples depleted of CD19-positive cells using Myltenii magnetic beads were also used in the assay. Following a 3-day incubation at 37° C./5% CO₂, 1 μCi H³-thymidine was added to each well and proliferation was counted 16 hours later. The assay was performed in triplicate and the results, shown in FIG. 3, are reported as Stimulation Index over the mean count obtained with cells of unstimulated naïve mice.

Results: Based on counts from about ten experiments using at least 3 animals per experiment, the number of cells in PLN of naïve mice was on average 5×10⁵ to 1×10⁶ cells per node compared to 1.5×10E6 to 8×10E6 cells per node in exosome-immunized mice. This increased number in cells was not observed in spleen cells, most likely because of the route of immunization used. FACS analysis of PLN cells revealed an average of 25.5+/−4.2% CD19+ cells in naïve PLN whereas 40+/−3.6% of the same cells was detected in exosome-immunized mice. Furthermore, as shown FIG. 3 panel A, PLN cells from exosome-immunized mice (1-PLN) were activated as indicated by their potency to proliferate even in the absence of stimulation in vitro. Both PLN cells from naïve (N-PLN) and immunized mice were stimulated by incubation with YAC exosomes. Higher SI was obtained with I-PLN cells more likely because these cells are already activated compared to the cells from the N-PLN group. This effect was exosome species specific since incubation with exosome derived from a human cell line (221) did not lead to proliferation above the level of unstimulated cells. This proliferation profile could also be detected with spleen cells (FIG. 3, panel B) although the stimulation indexes were much lower with these cells. Again, this is most likely because of the route of immunization used. Nevertheless, proliferation was abolished in all stimulation groups when CD19-depleted cells were used (1-S/CD19-) indication that B cell proliferation was measured in this assay.

Conclusion: Injection of YAC exosomes in mice induced activation of B cells in PLN and spleen. Naïve B cells could also be induced to proliferate when incubated with YAC exosomes but not human-derived exosomes. Therefore, YAC exosomes display adjuvant properties and may be used in vivo and in vitro to stimulate B cell.

### REFERENCES

1. Théry C, Zitvogel Z, Amigorena S. Exosomes: composition, biogenesis and function. Nat. Rev. Immunol. 2(8), 569-579 (2002).
2. Delcayre A., Shu H., Le Pecq J. B. Dendritic cell-derived Exosomes in Cancer Immunotherapy: Exploiting Nature's Antigen Delivery Pathway. Expert Rev. Anti-Cancer Therapy 5(3), in press (2005).
3. Zitvogel L, Regnault A, Lozier A et al. Eradication of established murine tumors using a novel cell-free vaccine: dendritic cell-derived exosomes. Nat. Med. 4(5), 594-600 (1998).
4. Morse M A, Garst J, Osada T, et al. A phase I study of dexosome immunotherapy in patients with advanced non-small cell lung cancer. J. Transl. Med. 3(1), 9 (2005).
5. Escudier B, Dorval T, Chaput N et al. Vaccination of metastatic melanoma patients with autologous dendritic cell (DC) derived exosomes: results of the first phase I clinical trial. J. Transl. Med. 3(1), 10 (2005).
6. Wolfers J, Lozier A, Raposo G et al. Tumor-derived exosomes are a source of shared tumor ejection antigens for cross-priming. Nat. Med. 7(3), 297-303 (2001).
7. Andre F, Schartz N E, Chaput N, Flament C, Raposo G, Amigorena S, Angevin E, Zitvogel L. Tumor-derived exosomes: a new source of tumor rejection antigens. Vaccine 20(Suppl 4), A28-31 (2002).
8. Andre F, Schartz N E, Movassagh M, Flament C, Pautier P, Morice P, Pomel C, Lhomme C, Escudier B, Le Chevalier T, Tursz T, Amigorena S, Raposo G, Angevin E, Zitvogel L. Malignant effusions and immunogenic tumour-derived exosomes. Lancet 360(9329), 295-305 (2002).
9. Altieri S L, Khan A N, Tomasi T B. Exosomes from plasmacytoma cells as a tumor vaccine. J. Immunother. 27(4), 282-8 (2004).
10. Taylor D D, Gercel-Taylor C. Tumour-derived exosomes and their role in cancer-associated T-cell signalling defects. Br. J. Cancer. 92(2), 305-11 (2005).
11. Peche H, Heslan M, Usal C, Amigorena S, Cuturi M C. Presentation of donor major histocompatibility complex antigens by bone marrow dendritic cell-derived exosomes modulates allograft rejection. Transplantation 76(10), 1503-10 (2003).
12. Andreola G, Rivoltini L, Castelli C, Huber V, Perego P, Deho P, Squarcina P, Accornero P, Lozupone F, Lugini L, Stringaro A, Molinari A, Arancia G, Gentile M, Parmiani G, Fais S. Induction of lymphocyte apoptosis by tumor cell secretion of FasL-bearing microvesicles. J. Exp. Med. 195(10), 1303-16 (2002).
13. Frangsmyr L, Baranov V, Nagaeva O, Stendahl U, Kjellberg L, Mincheva-Nilsson L. Cytoplasmic microvesicular form of Fas ligand in human early placenta: switching the tissue immune privilege hypothesis from cellular to vesicular level. Mol. Hum. Reprod. 11(1), 35-41 (2005).
14. Janowska-Wieczorek A, Wysoczynski M, Kijowski J, Marquez-Curtis L Machalinski B, Ratajczak J, Ratajczak M Z. Microvesicles derived from activated platelets induce metastasis and angiogenesis in lung cancer. Int. J. Cancer. 113(5), 752-60 (2005).
15. Hugel B, Martinez M C, Kunzelmann C, Freyssinet J M. Membrane microparticles: two sides of the coin. Physiology (Bethesda) 20(1), 22-27 (2005).
16. Whiteside T L. Tumour-derived exosomes or microvesicles: another mechanism of tumour escape from the host immune system? Br. J. Cancer 92(2), 209-11 (2005).
17. Gould S J, Booth A M, Hildreth J E. The Trojan exosome hypothesis. Proc. Natl. Acad. Sci. USA 100(19), 10592-7 (2003).
18. Nguyen D G, Booth A, Gould S J, Hildreth J E. Evidence that HIV budding in primary macrophages occurs through the exosome release pathway. J. Biol. Chem. 278(52), 52347-54 (2003).
18b. Sharova et al., the EMBO J. (2005) 1-9.
18c. Pelchen-Matthews et al., Journal of Cell Biology 162 (2005) 443-455
19. Morita E, Sundquist W I. Retrovirus budding. Annu. Rev. Cell Dev. Biol. 20, 395-425 (2004).
20. Masciopinto F, Giovani C, Campagnoli S. et al. Association of hepatitis C virus envelope proteins with exosomes. Eur. J. Immunol. 34(10), 2834-42 (2004).
21. Pelchen-Matthews A, Raposo G, Marsh M. Endosomes, exosomes and Trojan viruses. Trends Microbiol. 12(7), 310-6 (2004).
22. Flanagan J, Middeldorp J, Sculley T. Localization of the Epstein-Barr virus protein LMP 1 to exosomes. J. Gen. Virol. 84, 1871-9 (2003).
23. Gutwein P, Stoeck A, Riedle S et al. Cleavage of 11 in exosomes and apoptotic membrane vesicles released from ovarian carcinoma cells. Clin Cancer Res. 11 (7), 2492-501 (2005).
24. Taylor M R, Couto J R, Scallan C D, Ceriani R L, Peterson J A. Lactadherin (formerly BA46), a membrane-associated glycoprotein expressed in human milk and breast carcinomas, promotes Arg-Gly-Asp (RGD)-dependent cell adhesion. DNA Cell Biol. 16(7):861-9 (1997).
25. Silvestre, J. S., Théry, C., Hamard, G., Boddaert, J., Aguilar, B., Delcayre, A., Houbron, C., Tamarat, R., Clergue, M., Duriez, M., Merval, R., Lévy, B., Tedgui, A., Amigorena, S, and Mallat, Z. Lactadherin/MFG-E8: a novel angiogenic protein required for VEGF signalling. Nature Med. 11(5), 499-506 (2005).
26. Harlow E and Lane D. Antibodies: A laboratory manual. Cold Spring Harbor Laboratory, (1988).
27. Babcook, J. S.; Leslie, K. B.; Olsen, O. A.; Salmon, R. A. and Schrader, J. W. A novel strategy for generating monoclonal antibodies from single, isolated lymphocytes producing antibodies of defined specificities. Proc. Natl. Acad. Sci. U.S.A 93,7843-48 (1996).

1-38. (canceled)

39. A method for neutralizing exosomes in a subject, the method comprising administering to a subject in need thereof a neutralizing-effective amount of an exosome-specific ligand, said administration causing a neutralization of exosomes.

40. The method of claim 39, wherein the exosome-specific ligand is an antibody or a fragment or derivative thereof.

41. The method of claim 40, wherein the antibody is a monoclonal antibody.

42. The method of claim 40, wherein the antibody, or fragment or derivative thereof, comprises a heavy chain variable polypeptide sequence encoded by a nucleotide sequence selected from SEQ ID NOs: 1 to 12.

43. The method of claim 39, wherein the exosome-specific ligand is a protein, or a domain thereof, that selectively binds an exosome-specific marker.

44. The method of claim 43, wherein the protein is lactadherin or a fragment thereof comprising at least the C1 and/or C2 domain.

45. The method of claim 39, wherein the exosome-specific ligand is a chimeric polypeptide comprising an exosome-specific ligand-binding domain fused to a dimerization or multimerization domain, in particular to an Fc portion of an immunoglobulin.

46. The method of claim 39, wherein the exosome-specific ligand comprises several exosome-specific ligand-binding domains.

47. The method of claim 46, wherein the exosome-specific ligand comprises a protein, or a domain thereof, that selectively binds an exosome-specific marker, fused to an exosome-specific antibody or a fragment or derivative thereof.

48. A method for neutralizing a pathological immune response in a subject, the method comprising administering to a subject in need thereof a neutralizing-effective amount of an exosome-specific ligand, said administration causing a neutralization of said pathological immune response.

49. The method of claim 48, wherein said pathological immune response is selected from an allergic response, an inflammatory response, including host versus graft response, and an autoimmune response.

50. A method for neutralizing or treating (viral or microbial) infection in a subject, the method comprising administering to a subject in need thereof an amount of an exosome-specific ligand effective to neutralize exosomes in said subject, said administration causing a neutralization or treatment of said infection.

51. The method of claim 50, wherein the virus is selected from an immunodeficiency virus and a hepatitis virus.

52. A method for treating cancer (e.g., by reducing cancer immune evasion) in a subject, the method comprising administering to a subject in need thereof an amount of an exosome-specific ligand effective to neutralize exosomes in said subject, said administration causing a treatment of said cancer.

53. A composition comprising an exosome-specific ligand and a pharmaceutically acceptable carrier or excipient.

54. An antibody, or a fragment or derivative thereof having substantially the same antigen specificity, wherein said antibody selectively binds an exosome.

55. The antibody, or fragment or derivative thereof, of claim 54, which comprises a heavy chain variable polypeptide sequence encoded by a nucleotide sequence selected from SEQ ID NOs: 1 to 12.

56. A method of producing exosome-specific antibodies, comprising:
1) preparing exosomes from a non-human animal with a given genetic background;
2) immunizing a non-human animal with said vesicles, wherein animals for immunization and preparation of vesicles share the same or very similar genetic background; and
3) collecting, from the immunized animal, antibodies reacting with the vesicles of step 1 or corresponding antibody-producing cells.

57. A method of isolating exosome-specific ligands, comprising:
1) preparing exosomes,
2) screening libraries of compounds reacting with said vesicles,
3) selecting exosome ligands from step 2 that do not react with the exosome-producing cell.

58. A method of stimulating B cells in vivo, comprising administering to an animal or a subject in need thereof an amount of exosomes sufficient to cause a stimulation of B cells in said animal or subject.
